(19) United States
(12) Plant Patent Application Publication
(10) Pub. No.: US 2002/0073470 P1
(43) Pub. Date: Jun. 13, 2002

(54) Variety of geranium plant named 'Pennea'

(76) Inventor: Andrea Michalik, Dresden (DE)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 09/733,016

(22) Filed: Dec. 8, 2000

(51) Int. Cl. A01H 5/00

(52) U.S. Cl. PLT/332

(57) ABSTRACT

A new and distinct ivy geranium plant with shiny purple colored flowers above medium green foliage. The plant is suitable for planting in hanging baskets.

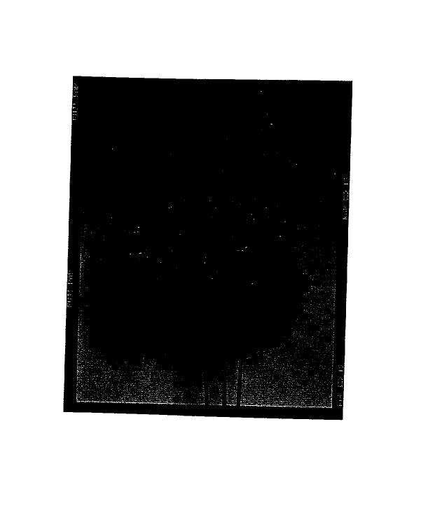

### BACKGROUND OF THE INVENTION

The present invention comprises a new and distinct cultivar of Pelargonium peltatum known by the varietal name ‘Pennea’. The new variety was discovered in 1996 in a selective breeding program in Dresden, Germany. The new variety is the result of a combination breeding and embryo rescue. The new variety was first asexually reproduced in 1997 by cuttings in Dresden, Germany. The new variety has been trial and field tested and has been found to retain its distinctive characteristics and remain true to type through successive propagations.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Dresden, Germany. The color readings were taken outdoors in shadow during summer. Color references are primarily to The R.H.S. Colour Chart of The Royal Horticultural Society of London.

Plant:

Classification.—Botanical: Pelargonium peltatum. Commercial: Ivy geranium.

Form.—Hanging.

Stem length.—70-120 cm.

Strength.—Does not require artificial support.

Response time.—4 weeks.

Leaves.—Size: 8-10 cm long. Shape: Ivy-shaped. Margin: Emarginate. Texture: Hairy. Color: Green Group 137A. Ribs and veins: Color: Brighter than leaf surface.

Petioles.—Color: Medium green. Length: 3-4 cm.

Stem/branches.—Color: Medium green. Internode length: 4-6 cm.

The bud:

Shape when just showing color.—Overall: Semi-spherical. Individual bud: Elliptical.

Color.—Green Group 139B.

Size when just showing color.—Umbel: 7-9 cm across. Individual bud: 1.0-1.5 cm long; 1.0 cm wide.

Inflorescence:

Blooming habit.—Double.

Size of fully open bloom.—5-6 cm across.

Borne.—Flower on umbel; umbel on pedicel; pedicel on peduncle.

Flowers.—Form: Zygomorph. Number of petals: 24. Size: 1-2.5 cm across. Petals: Color: Upper surface: Red-Purple Group 66B Lower surface: Red-Purple Group 74B Texture and appearance: Velvety.

Petaloids.—Absent.

Pedicel.—Length: 2.0-2.5 cm. Color: Medium green.

Peduncle.—Length: 9.0-11.0 cm. Color: Medium green.

Disease/pest resistance.—No unusual susceptibility to diseases or pests has been noted to date.

Lasting Quality.—Long-lasting; 2 weeks per umbel

Reproductive organs:

Stamens.—Anthers: 3 mm long. Filaments: Length: 0.5 cm maximum. Color: Bright pink. Pollen: Yellow.

Pistils.—Number: Five. Length: 0.3-0.4 cm. Stigma: Pink in color. Style: 0.8 cm long.

Ovaries.—Color: Greenish. Length: 0.4 cm.

Fruit.—Very rarely present.

### GENERAL CHARACTERISTICS

1. Very intense and shiny purple flower color.

2. Early flowering.

3. Vigorous growth habit.

4. Suitable for hanging baskets.

I claim:

1. A new and distinct variety of geranium plant substantially as shown and described.
